(19) United States
(12) Patent Application Publication
(10) Pub. No.: US 2024/0065533 A1
(43) Pub. Date: Feb. 29, 2024

(54) ENDOSCOPE WITH INTERCHANGEABLE CANNULA ADAPTER

(71) Applicant: Integrated Endoscopy, Inc., Irvine, CA (US)

(72) Inventors: Bradley Jay Sharp, Irvine, CA (US); Andrew Ryan Sharp, Irvine, CA (US)

(21) Appl. No.: 17/823,487

(22) Filed: Aug. 30, 2022

(51) Int. Cl. A61B 1/00; A61B 1/018

(52) U.S. Cl. CPC A61B 1/00128; A61B 1/00105; A61B 1/018

(57) ABSTRACT

An endoscope assembly system includes a housing and an adapter that couples to the housing. The adapter has a bore that receives an endoscope tube therethrough. The adapter has a cannula connector on an outer surface of the adapter that allows it to couple with a cannula positioned over at least a portion of the adapter. Different adapters can be coupled to the housing to allow the endoscope to be used with a variety of different cannulas having different cannula locking mechanisms, thereby allowing one endoscope assembly to be used with a variety of different cannula designs.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND OF THE INVENTION

### Field

The present disclosure is directed to an endoscope assembly, and more particularly to an endoscope assembly with different interchangeable adapters that allow the endoscope assembly to be coupled to cannulas having different locking mechanisms.

### Description of the Related Art

Endoscopes are widely used in medical procedures. In operation, the endoscope is inserted through an access cannula that has been inserted into the patient (e.g., via an incision), and the endoscope is locked to the cannula via the cannula's locking mechanism that couples to a portion of the endoscope. Various cannula manufacturers provided cannulas with different locking mechanisms, requiring different endoscopes to mate with the particular cannula design.

### SUMMARY

Accordingly, there is a need for an endoscope design that can be utilized with a variety of different cannulas having different cannula locking mechanisms. In accordance with one aspect, an endoscope assembly (e.g., rigid endoscope) having one or more adapters is provided, thereby providing a “universal” endoscope assembly that can lockingly couple with a variety of different cannulas having different cannula locking mechanisms. Optionally, the endoscope assembly can be a single use assembly.

In accordance with one aspect, and endoscope assembly is provided. The endoscope assembly comprises a housing and an adapter configured to couple to the housing. The adapter has a bore configured to receive an endoscope tube therethrough. The adapter has a cannula connector on an outer surface of the adapter that is configured to removably couple with a cannula positioned over at least a portion of the adapter. The adapter is interchangeable between a plurality of adapters configured to couple with a variety of different cannulas. Optionally, the adapter is removably coupleable to the housing.

In accordance with another aspect, an endoscope kit is provided. The kit comprises a housing and a plurality of interchangeable adapters configured to be coupled to the housing. Each adapter comprises a bore configured to receive an endoscope tube therethrough and a cannula connector on an outer surface of the adapter that is configured to couple with a cannula positioned over at least a portion of the adapter. The endoscope tube is configured to extend through the bore of the adapter. At least two of the plurality of interchangeable adapters are configured to lockingly couple with a different cannula. Optionally, each of the plurality of interchangeable adapters is configured to lockingly couple with a different cannula. Optionally, at least one of the plurality of interchangeable adapters is removably coupleable to the housing.

### EXAMPLES

In one example, an endoscope assembly can include a housing and an adapter configured to couple to the housing, the adapter having a bore configured to receive an endoscope tube therethrough, the adapter having a cannula connector on an outer surface of the adapter that is configured to removably couple with a cannula positioned over at least a portion of the adapter. The adapter is interchangeable between a plurality of adapters configured to couple with a variety of different cannulas.

Some examples include the assembly of any one of the preceding examples, wherein the adapter has one or more latching connectors at a proximal end of the adapter, the adapter coupling with the housing at least in part via the latching connectors.

Some examples include the assembly of any one of the preceding examples, wherein the housing comprises one or more openings configured to lockingly receive the one or more latching connectors therethrough.

Some examples include the assembly of any one of the preceding examples, wherein the housing has a recessed portion in the outer surface that is configured to receive at least a portion of a flange of the adapter, the flange disposed proximal of the cannula connector.

Some examples include the assembly of any one of the preceding examples, wherein the flange is configured to extend into the recessed portion so that an outer surface of the flange is flush with the outer surface of the housing adjacent the recessed portion.

Some examples include the assembly of any one of the preceding examples, further comprising a gasket interposed between a surface of the housing and a surface of the adapter, the gasket configured to seal the adapter relative to the tube of the endoscope.

Some examples include the assembly of any of the preceding examples, wherein the cannula connector comprises a circumferential groove defined on an outer surface of the adapter, the groove configured to removably lock with a cannula when the cannula connector is inserted in a proximal end of the cannula.

In one example, an endoscope kit includes an endoscope comprising an endoscope tube, a housing configured to extend over at least a portion of the endoscope so that the endoscope tube extends through and opening in the housing, and a plurality of interchangeable adapters configured to be coupled to the housing. Each adapter comprises a bore configured to receive the endoscope tube therethrough, and a cannula connector on an outer surface of the adapter that is configured to couple with a cannula positioned over at least a portion of the adapter. The endoscope tube is configured to extend through the bore of the adapter, at least two of the plurality of interchangeable adapters being configured to lockingly couple with a different cannula.

Some examples include the kit of any preceding example, wherein each of the plurality of interchangeable adapters are configured to lockingly couple with a different cannula.

Some examples include the kit of any preceding example, wherein at least one of the plurality of interchangeable adapters is removably coupleable to the housing.

Some examples include the kit of any preceding example, wherein the housing has a recessed portion configured to receive at least a portion of the adapter therein.

Some examples include the kit of any preceding example, wherein the adapter has a flange adjacent the cannula connector configured to at least partially extend into the recessed portion when the adapter is coupled to the housing.

Some examples include the kit of any preceding example, wherein the housing has one or more openings configured to receive one or more latching connectors of the adapter therein to lockingly couple the adapter to the housing.

Some examples include the kit of any preceding example, further comprising a gasket interposed between a surface of the housing and a surface of the adapter, the gasket configured to seal the adapter relative to the tube of the endoscope.

Some examples include the kit of any preceding example, wherein the cannula connector comprises a circumferential groove defined on an outer surface of the adapter, the groove configured to removably lock with a proximal key connector of the cannula when the cannula connector is inserted in the proximal end of the cannula.

In one example, an interchangeable adapter for an endoscope assembly can have an adapter body. The adapter body is configured to couple to a housing of the endoscope assembly, the adapter having a bore configured to receive an endoscope tube therethrough, the adapter having a cannula connector on an outer surface of the adapter that is configured to removably couple with a cannula when the cannula is positioned over at least a portion of the adapter.

Some examples include the adapter of any preceding example, further comprising means for coupling the adapter body with the housing.

Some examples include the adapter of any preceding example, further comprising one or more latching connectors at a proximal end of the adapter body, the adapter body configured to couple with the housing at least in part via the latching connectors.

Some examples include the adapter of any preceding example, wherein the adapter body further comprises a flange disposed proximal of the cannula connector.

Some examples include the adapter of any preceding example, wherein the flange is configured to extend into a recessed portion of the housing so that an outer surface of the flange is flush with the outer surface of the housing adjacent the recessed portion.

Some examples include the adapter of any preceding example, further comprising a gasket attached to a surface of the adapter, where the gasket is interposed between a surface of the housing and a surface of the adapter when the adapter is coupled to the housing, the gasket configured to seal the adapter relative to a tube of the endoscope.

Some examples include the adapter of any preceding example, wherein the cannula connector comprises a circumferential groove defined on an outer surface of the adapter, the circumferential groove configured to removably lock with a cannula connector of a cannula.

### BRIEF DESCRIPTION OF THE DRAWINGS

FIG. 1 is a schematic side view of an endoscope with an interchangeable cannula connector.

FIG. 2A is a partial schematic cross-sectional view of a housing of the endoscope coupled with a cannula connector.

FIG. 2B is a schematic side view of the cannula connector of FIG. 2A.

FIG. 3A is a partial schematic cross-sectional view of a housing of the endoscope coupled with a cannula connector.

FIG. 3B is a schematic side view of the cannula connector of FIG. 3A.

FIG. 4A is a partial schematic cross-sectional view of a housing of the endoscope coupled with a cannula connector.

FIG. 4B is a schematic side view of the cannula connector of FIG. 4A.

FIG. 5A is a partial schematic cross-sectional view of a housing of the endoscope coupled with a cannula connector.

FIG. 5B is a schematic side view of the cannula connector of FIG. 5A.

FIG. 6A is a partial schematic cross-sectional view of a housing of the endoscope coupled with a cannula connector.

FIG. 6B is a schematic side view of the cannula connector of FIG. 6A.

FIG. 7A is a partial schematic cross-sectional view of a housing of the endoscope coupled with a cannula connector.

FIG. 7B is a schematic side view of the cannula connector of FIG. 7A.

FIG. 8 is a schematic exploded view of a portion of the housing of the endoscope and a cannula connector.

FIG. 9 is a schematic partial cross-sectional view of a cannula slid over the endoscope tube and cannula connector during coupling of the cannula with the endoscope.

FIG. 10 is a schematic view of an endoscope kit with interchangeable cannula connectors.

FIG. 11 is a block diagram of a method of using an endoscope with interchangeable cannula connectors.

FIGS. 12A-12B show a perspective view and a cross-sectional view of a portion of a cannula adapter.

FIGS. 13A-13B show a left side view and a right side view of a portion of a cannula adapter.

FIG. 14 shows a perspective view of a portion of a cannula adapter.

FIG. 15 shows a perspective view of a portion of a cannula adapter.

### DETAILED DESCRIPTION

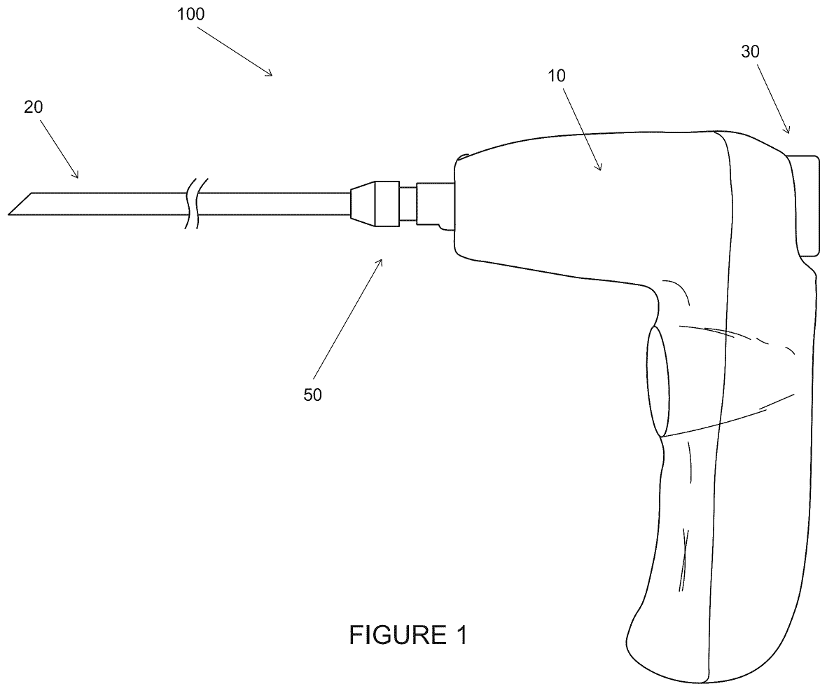

FIG. 1 shows an endoscope 100 having a housing 10, an endoscope tube 20 (e.g., a rigid endoscope tube), an eyepiece 30, and an adapter 50. In some implementations, the endoscope 100 can be disposable, portable and/or handheld. In some designs, the housing 10 can house one or more batteries and electronics to power a light source (e.g., in the endoscope 100) that can, for example, deliver light through the distal end of the endoscope tube 20. The adapter 50 can couple with a corresponding connector in a cannula that slides over the adapter 50, as described further below.

The adapter 50 can be a separate component that can be coupled to the distal portion of the housing 10. In one variation, the adapter 50 can have one or more male coupling members that couple with and insert into one or more female coupling members of the housing 10. In another variation, the adapter 50 can have one or more female coupling members that receive and couple to one or more male coupling members of the housing 10. In one variation, the adapter 50 is removably coupleable to the housing 10. In another variation, the adapter 50, once attached to the housing 10, is permanently fixed thereto.

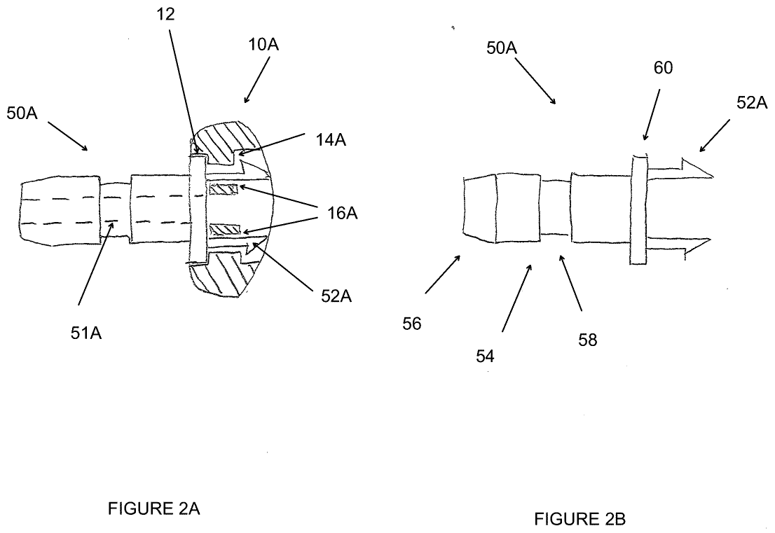

FIGS. 2A-2B show one variation of a cannula adapter 50A. The adapter 50A has one or more (e.g., a pair of) male coupling members 52A that can be received in one or more (e.g., a pair of) openings 16A in the housing 10A. For simplicity, only a distal portion of the housing 10A is shown. However, one skilled in the art will recognize that the rest of the housing 10A may be similar to or different than the housing 10 shown in FIG. 1.

As shown in FIG. 2B, the one or more coupling member 52A can be a pair of barbed latch prongs. However, the adapter 50A can have fewer or more than two such barbed latch prongs. The adapter 50A also has a cannula coupling member 54 that optionally includes a tapered distal portion 56 and a recessed or circumferential slot portion 58. The adapter 50A optionally includes a flange 60 between the cannula coupling member 54 and the barbed latch prongs 52A. The adapter 50A has a bore 51A that extends therethrough (see dashed lines in FIG. 2A) sized to receive the endoscope tube 20 therethrough, as further discussed below.

As shown in FIG. 2A, the cannula adapter 50A is coupled to the housing 10A (e.g., to a distal portion of the housing 10A) so that the barbed latch prongs 52A extend through the openings 16A in the housing 10A and catch (e.g., bear) against tabs or ledges 14A in the housing 10A. Optionally, an audible feedback (e.g., a “clicking” or “snapping” sound) is provided when the cannula adapter 50A is coupled with the housing 10A to indicate to the medical provider that the coupling of the adapter 50A with the housing 10A has been completed. Optionally, the tabs or ledges 14A can inhibit removal of the cannula adapter 50A from the housing 10A once coupled thereto. Optionally, the housing 10A has a recess 12 that receives at least a portion of the flange 60 when the cannula adapter 50A is coupled to the housing 10A. Optionally, the recess 12 receives the flange 60 so that an outer surface of the flange 60 is flush with an outer surface of the housing 10A that is adjacent the flange 60. In another variation, the recess 12 is excluded and the flange 60 attaches to an outer surface of the housing 10A so that the flange 60 protrudes from such outer surface of the housing 10A.

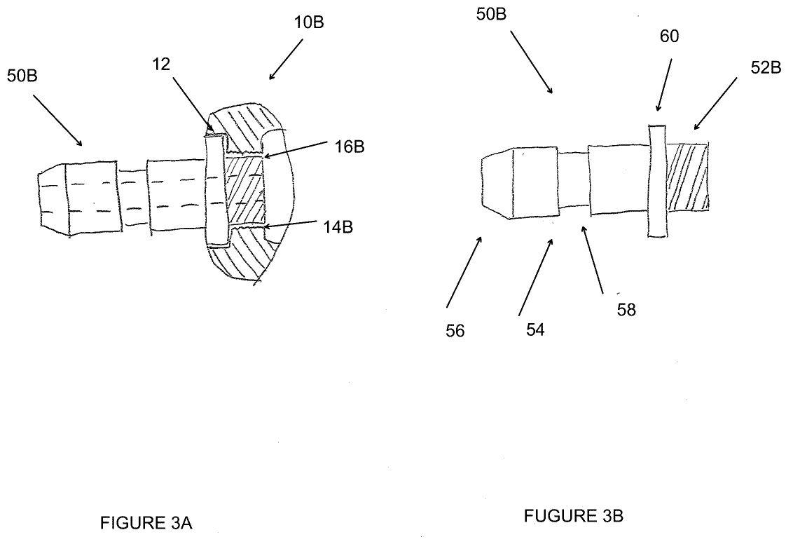

FIGS. 3A-3B schematically illustrate a cannula adapter 50B on its own and coupled to a housing 10B of an endoscope, such as the endoscope 100. Some of the features of the cannula adapter 50B and housing 10B are similar to features in the cannula adapter 10A and housing 10A in FIGS. 2A-2B. Thus, references numerals used to designate the various components of the cannula adapter 50B and housing 10B are identical to those used for identifying the corresponding components of the cannula adapter 50A and housing 10A in FIGS. 2A-2B, except that a “B” is added to the numerical identifier. Therefore, the structure and description for the various features of the cannula adapter 50A and housing 10A in FIGS. 2A-2B are understood to also apply to the corresponding features of the cannula adapter 50B and housing 10B in FIGS. 3A-3B, except as described below.

The cannula adapter 50B differs from the cannula adapter 50A in that it has a male coupling member 52B in the form of a threaded adapter 52B. The male thread of the threaded adapter 52B can extend into an opening 16B in the housing 10B and threadably couple with a female threaded portion 14B of the housing 10B to couple the cannula adapter 50B to the housing 10B. For simplicity, only a distal portion of the housing 10B is shown. However, one skilled in the art will recognize that the rest of the housing 10B may or may not be similar to the housing 10 shown in FIG. 1.

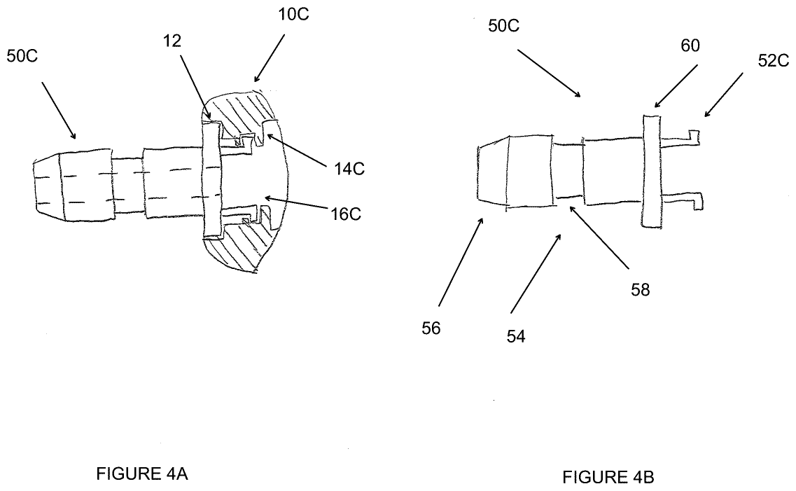

FIGS. 4A-4B schematically illustrate a cannula adapter 50C on its own and coupled to a housing 10C of an endoscope, such as the endoscope 100. Some of the features of the cannula adapter 50C and housing 10C are similar to features in the cannula adapter 10A and housing 10A in FIGS. 2A-2B. Thus, references numerals used to designate the various components of the cannula adapter 50C and housing 10C are identical to those used for identifying the corresponding components of the cannula adapter 50A and housing 10A in FIGS. 2A-2B, except that a “C” is added to the numerical identifier. Therefore, the structure and description for the various features of the cannula adapter 50A and housing 10A in FIGS. 2A-2B are understood to also apply to the corresponding features of the cannula adapter 50C and housing 10C in FIGS. 4A-4B, except as described below.

The cannula adapter 50C differs from the cannula adapter 50A in that it has a pair of male coupling member 52C with key elements that can lock the cannula adapter 50C to the housing 10C. The key elements can extend into the opening 16C in the housing 10C and the adapter 50C rotated so that the key elements travel along slots 14C in the housing 10C to couple the cannula adapter 50C to the housing 10C. Optionally, the cannula adapter 50C can be decoupled from the housing 10C by rotating the cannula adapter 50C in the opposite direction until the key elements rotate to the location of an opening in the slots 14C that allows the axial separation of the cannula adapter 50C from the housing 10C. For simplicity, only a distal portion of the housing 10C is shown. However, one skilled in the art will recognize that the rest of the housing 10C may or may not be similar to the housing 10 shown in FIG. 1.

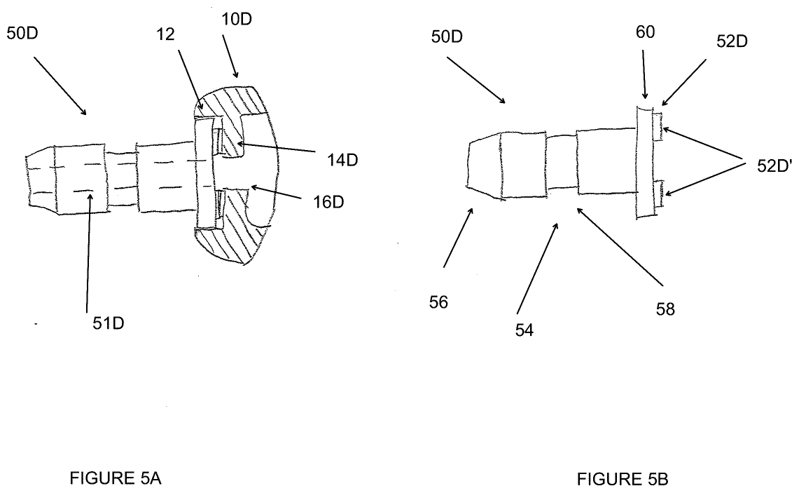

FIGS. 5A-5B schematically illustrate a cannula adapter 50D on its own and coupled to a housing 10D of an endoscope, such as the endoscope 100. Some of the features of the cannula adapter 50D and housing 10D are similar to features in the cannula adapter 10A and housing 10A in FIGS. 2A-2B. Thus, references numerals used to designate the various components of the cannula adapter 50D and housing 10D are identical to those used for identifying the corresponding components of the cannula adapter 50A and housing 10A in FIGS. 2A-2B, except that a “D” is added to the numerical identifier. Therefore, the structure and description for the various features of the cannula adapter 50A and housing 10A in FIGS. 2A-2B are understood to also apply to the corresponding features of the cannula adapter 50D and housing 10D in FIGS. 5A-5B, except as described below.

The cannula adapter 50D differs from the cannula adapter 50A in that it has a one or more coupling member 52D in the form of one or more magnets 52D′. The one or more magnets 52D′ can releasably couple with one or more portions 14D in the housing 10D that have an opposite polarity (e.g., one or more magnets with opposite polarity attached to a surface of the housing 10D, or one or more metal portions on a surface of the housing 10D) to couple the cannula adapter 50D to the housing 10D. The adapter 50D couples to the housing 10D (see FIG. 5A) so that the bore 51D of the adapter 50D substantially aligns with an opening 16D in the housing 10D. For simplicity, only a distal portion of the housing 10D is shown. However, one skilled in the art will recognize that the rest of the housing 10D may or may not be similar to the housing 10 shown in FIG. 1.

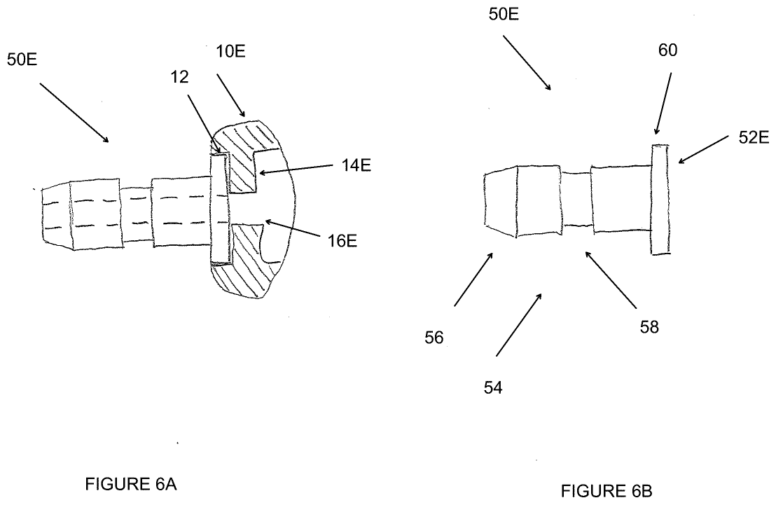

FIGS. 6A-6B schematically illustrate a cannula adapter 50E on its own and coupled to a housing 10E of an endoscope, such as the endoscope 100. Some of the features of the cannula adapter 50E and housing 10E are similar to features in the cannula adapter 10A and housing 10A in FIGS. 2A-2B. Thus, references numerals used to designate the various components of the cannula adapter 50E and housing 10E are identical to those used for identifying the corresponding components of the cannula adapter 50A and housing 10A in FIGS. 2A-2B, except that an “E” is added to the numerical identifier. Therefore, the structure and description for the various features of the cannula adapter 50A and housing 10A in FIGS. 2A-2B are understood to also apply to the corresponding features of the cannula adapter 50E and housing 10E in FIGS. 6A-6B, except as described below.

The cannula adapter 50E differs from the cannula adapter 50A in that its coupling member 52E is a portion of the flange 60 that is press-fit into the recess 12 and against a ledge 14E of the housing 10E to couple the cannula adapter 50E to the housing 10E. An opening 16E in the housing 10E allows the endoscope tube 20 to pass through the housing 10E and the cannula adapter 50E once coupled thereto. For simplicity, only a distal portion of the housing 10E is shown. However, one skilled in the art will recognize that the rest of the housing 10E may or may not be similar to the housing 10 shown in FIG. 1.

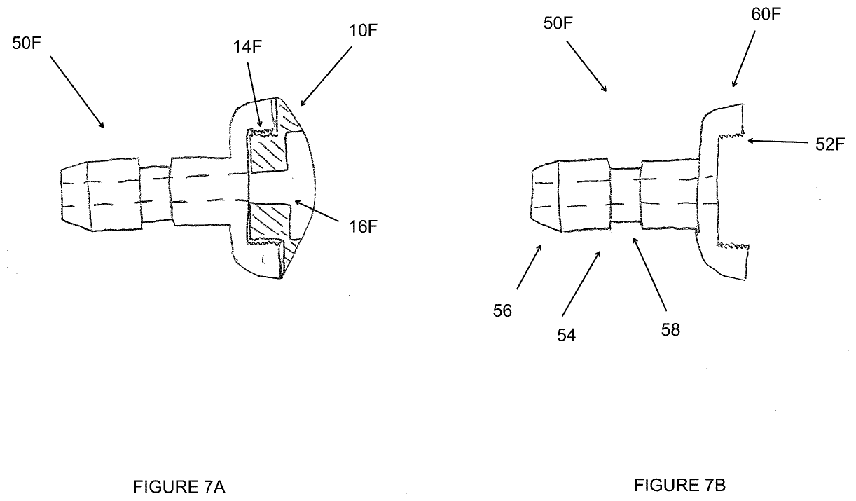

FIGS. 7A-7B schematically illustrate a cannula adapter 50F on its own and coupled to a housing 10F of an endoscope, such as the endoscope 100. Some of the features of the cannula adapter 50F and housing 10F are similar to features in the cannula adapter 10A and housing 10A in FIGS. 2A-2B. Thus, references numerals used to designate the various components of the cannula adapter 50F and housing 10F are identical to those used for identifying the corresponding components of the cannula adapter 50A and housing 10A in FIGS. 2A-2B, except that an “F” is added to the numerical identifier. Therefore, the structure and description for the various features of the cannula adapter 50A and housing 10A in FIGS. 2A-2B are understood to also apply to the corresponding features of the cannula adapter 50F and housing 10F in FIGS. 6A-6B, except as described below.

The cannula adapter 50F differs from the cannula adapter 50A in that its coupling member 52F is a female threaded portion 52F that threadably couples with a male threaded portion 14F of the housing 10F to couple the cannula adapter 50F to the housing 10F. Optionally, the threaded connection between the cannula adapter 50F and the housing 10F allows the adapter 50F to be removably coupled to the housing 10F. Optionally, the female threaded portion 52F is defined at least in part in the flange 60F of the cannula adapter 50F. Optionally, the flange 60F aligns with the housing 10F when coupled thereto (e.g., so that an outer surface of the flange 60F aligns with an outer surface of the housing 10F that is adjacent the flange 60F). An opening 16F in the housing 10F allows the endoscope tube 20 to pass through the housing 10F and the cannula adapter 50F once coupled thereto. For simplicity, only a distal portion of the housing 10F is shown. However, one skilled in the art will recognize that the rest of the housing 10F may or may not be similar to the housing 10 shown in FIG. 1.

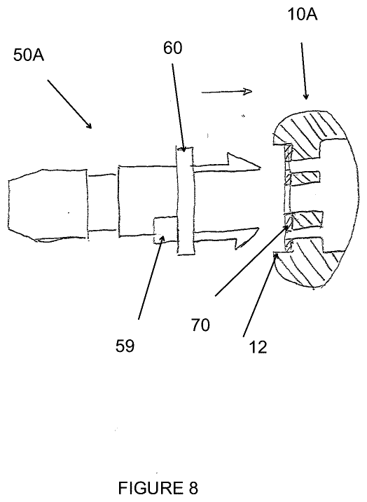

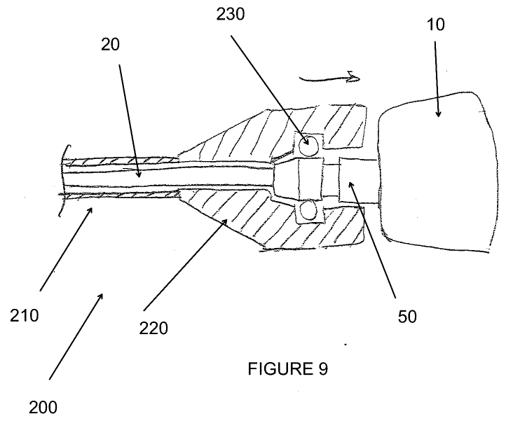

FIG. 8 shows a variation of the cannula adapter 50A and housing 10A. The adapter 50A can optionally have a key portion 59 on a surface thereof for registering with the cannula, once the cannula is slid over the cannula adapter 50A and coupled thereto. Optionally, key portion 59 allows the cannula to couple to the cannula adapter 50A in a particular rotational orientation. Optionally, a gasket 70 can be interposed between the cannula adapter 50A and the housing 10A (e.g., can be disposed in the recess 12 so that it is in between the flange 60 of the cannula adapter 50A and a base surface of the recess 12. The gasket 70 can optionally seal the cannula adapter 50A to the endoscope tube 20 when the adapter 50A is coupled to the housing 10A (e.g., moved in direction of arrow in FIG. 8). Optionally, the gasket 70 is a pressure cuff. For simplicity, only a distal portion of the housing 10A is shown. However, one skilled in the art will recognize that the rest of the housing 10A may or may not be similar to the housing 10 shown in FIG. 1.

FIG. 9 shows a cannula 200 having a cannula tube 210 and a connector 220 slid over the endoscope tube 20 and cannula adapter 50 during a coupling operation of the cannula 200 to the adapter 50. The connector 220 can have one or more seals 230 that can lockingly engage the recessed or circumferential slot portion 58 of the cannula adapter 50 once the connector 220 is fully coupled with the cannula adapter 50. Optionally, the cannula adapter 50 can have a key portion, such as the key portion 59 (see FIG. 8), to register and couple with the cannula 200 in a particular rotational orientation. Optionally, the connector 220 engages the cannula adapter 50 so that a proximal surface of the connector 220 is adjacent (e.g., in contact with) a distal surface of the housing 10. For simplicity, only a distal portion of the housing 10 is shown. However, one skilled in the art will recognize that the rest of the housing 10 in FIG. 8 may or may not be similar to the housing 10 shown in FIG. 1.

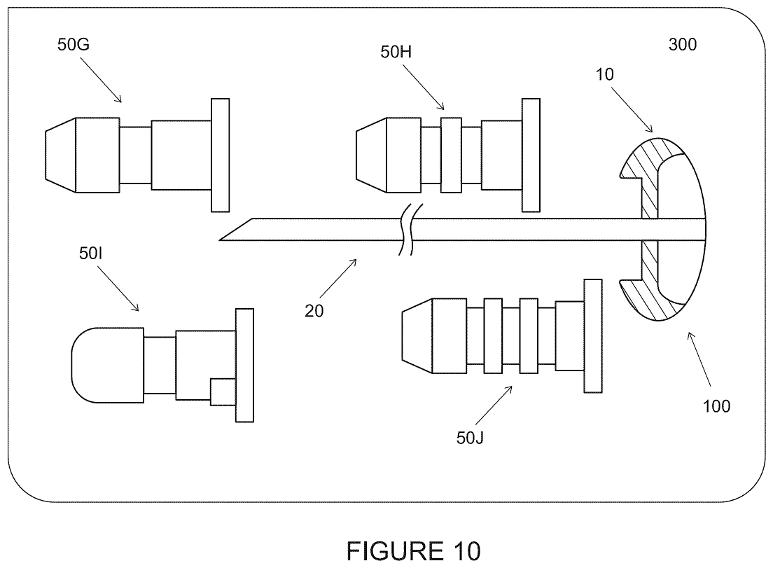

FIG. 10 is a schematic view of an endoscope kit 300. The kit 300 can include one or more endoscopes 100, each having a housing 10 and endoscope tube 20, as discussed above. The kit 300 can have one or more (e.g., a plurality of) separate cannula adapters 50G, 50H 50I 50J decoupled from the housing 10. The one or more endoscopes 100 and one or more separate cannula adapters 50G, 50H, 50I, 50J can optionally be provided in a single package (e.g., a sterilized or inert package), ready for use in a medical procedure. Optionally, one or more of the cannula adapters 50G, 50H 50I 50J can have a different cannula coupling member 54 for coupling with a different cannula design. Accordingly, the kit 300 advantageously allows the endoscope 100 to be used with (e.g., couple with) different cannula designs (e.g., from different cannula manufacturers) by coupling the cannula adapter 50G, 50H 50I 50J that corresponds to the particular cannula to the housing 10 to complete the endoscope assembly. Though FIG. 10 shows the kit 300 as having four different cannula adapters 50G, 50H 50I 50J, the invention contemplates the kit 300 having more or fewer adapters. In one variation, at least two of the cannula adapters are of the same type (e.g., to couple to the same cannula design). In another variation, each of the cannula adapters in the kit 300 can couple with a different cannula design. In still another variation, the kit can exclude the endoscope 100 and instead be a package (e.g., sterilized or inert package) of only the one or more (e.g., a plurality of cannula adapters), such as when a replacement kit of adapters is provided to a medical provider that already has an endoscope 100.

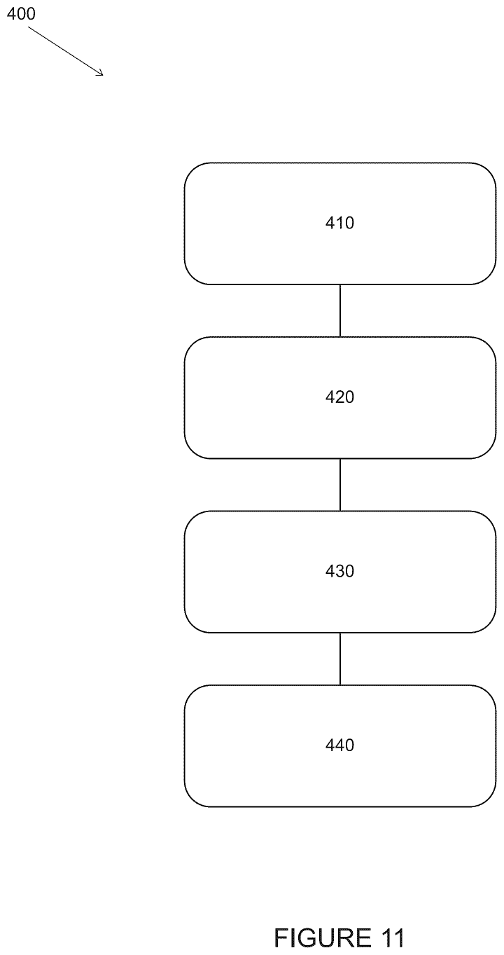

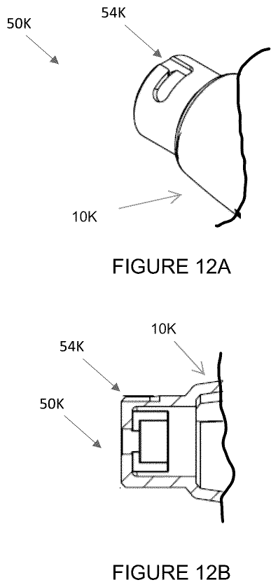

FIG. 11 shows a block diagram of a method 400 for assembling an endoscope (such as using the kit 300). The medical service provider (e.g., nurse, surgeon, physician's assistant) would first identify 410 the type of cannula to be used in the medical procedure, and select 420 the appropriate cannula adapter 50G, 50H 50I 50J from the kit 300 that corresponds to that cannula. The medical service provider would then slide 430 the cannula adapter over the endoscope tube 20 and couple the adapter to the housing 10 of the endoscope 100, thereby completing the endoscope assembly. The endoscope 100 could then be slid through the cannula to couple 440 the endoscope 100 to the cannula 200 (e.g., by coupling the connector 220 to the cannula adapter 50, as shown in FIG. 9). Optionally, the endoscope 100 is a single use endoscope, and would be discarded after use in the medical procedure. In another variation, the endoscope 100 can be a reusable endoscope that can be sterilized after use, allowing it to be used in subsequent medical procedures. Though not shown in FIG. 11, the kit 300 can additionally or alternatively include one or more of the cannula adapter 50K, 50L, 50M, 50N shown in FIGS. 12A-15. The cannula adapters in the kit 300 can be interchangeable.

The endoscope assembly and kit described herein advantageously provide a universal endoscope that can be used with a variety of cannula designs that have a different locking mechanism. Such an endoscope assembly and kit advantageously allows a hospital or other medical facility to reduce inventory, and therefore cost, in having to purchase different endoscope types that mate with different cannula designs. Additionally, as a single use device, the endoscope 100 allows a hospital or medical facility to eliminate costs associated with the sterilization and maintenance/storage of endoscopes.

FIGS. 12A-12B show a portion of a cannula adapter 50K. Some of the features of the cannula adapter 50K are similar to features of the cannula adapter 50A-50J in FIG. 2A-10. Thus, reference numerals used to designate the various components of the cannula adapter 50K are identical to those used for identifying the corresponding components of the cannula adapter 50A-50J in FIGS. 2A-10, except that a “K” has been added to the numerical identifier. Therefore, the structure and description for the various features of the cannula adapter 50A-50J and how it's operated and controlled in FIGS. 2A-10, including how they are coupled to the housing of the endoscope are understood to also apply to the corresponding features of the cannula adapter 50K in FIGS. 12A-12B, except as described below.

The cannula adapter 50K differs from the cannula adapters 50A-50J in that the cannula coupling member 54K has a key-slot that can receive a key (e.g., protrusion, post, peg) attached to the connector on the cannula to couple the cannula adapter 50K to the cannula.

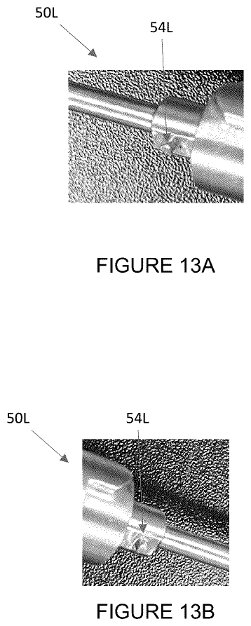

FIGS. 13A-13B show a portion of a cannula adapter 50L. Some of the features of the cannula adapter 50L are similar to features of the cannula adapter 50A-50K in FIG. 2A-10, 12A-12B. Thus, reference numerals used to designate the various components of the cannula adapter SOL are identical to those used for identifying the corresponding components of the cannula adapter 50A-50K in FIGS. 2A-10, 12A-12B, except that an “L” has been added to the numerical identifier. Therefore, the structure and description for the various features of the cannula adapter 50A-50K and how it's operated and controlled in FIGS. 2A-10, 12A-12B, including how they are coupled to the housing of the endoscope are understood to also apply to the corresponding features of the cannula adapter SOL in FIGS. 13A-13B, except as described below.

The cannula adapter SOL differs from the cannula adapters 50A-50K in that the cannula coupling member 54L has a pair of posts (e.g., keys) extending from opposite sides that can couple to key-slot(s) of the connector of the cannula to couple the cannula adapter SOL to the cannula.

FIG. 14 shows a portion of a cannula adapter 50M. Some of the features of the cannula adapter 50M are similar to features of the cannula adapter 50A-50L in FIG. 2A-10, 12A-13B. Thus, reference numerals used to designate the various components of the cannula adapter 50M are identical to those used for identifying the corresponding components of the cannula adapter 50A-50L in FIGS. 2A-10, 12A-13B, except that an “M” has been added to the numerical identifier. Therefore, the structure and description for the various features of the cannula adapter 50A-50L and how it's operated and controlled in FIGS. 2A-10, 12A-13B, including how they are coupled to the housing of the endoscope are understood to also apply to the corresponding features of the cannula adapter 50M in FIG. 14, except as described below.

The cannula adapter 50M differs from the cannula adapters 50A-50L in that the cannula coupling member 54M has one or more linear slots or recesses that can receive corresponding protrusions on the connector of the cannula to couple the cannula adapter 50M to the cannula.

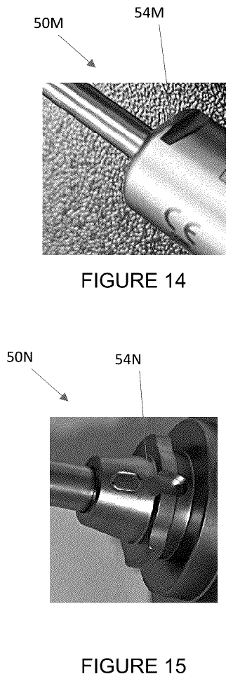

FIG. 15 shows a portion of a cannula adapter 50N. Some of the features of the cannula adapter 50N are similar to features of the cannula adapter 50A-50M in FIG. 2A-10, 12A-14. Thus, reference numerals used to designate the various components of the cannula adapter 50N are identical to those used for identifying the corresponding components of the cannula adapter 50A-50M in FIGS. 2A-10, 12A-14, except that an “N” has been added to the numerical identifier. Therefore, the structure and description for the various features of the cannula adapter 50A-50M and how it's operated and controlled in FIGS. 2A-10, 12A-14, including how they are coupled to the housing of the endoscope are understood to also apply to the corresponding features of the cannula adapter 50N in FIG. 15, except as described below.

The cannula adapter 50N differs from the cannula adapters 50A-5ML in that the cannula coupling member 54N has one or more slots or recesses in a disk that can receive corresponding protrusions on the connector of the cannula to couple the cannula adapter 50N to the cannula.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. An endoscope assembly, comprising:
a housing; and
an adapter configured to couple to the housing, the adapter having a bore configured to receive an endoscope tube therethrough, the adapter having a cannula connector on an outer surface of the adapter that is configured to removably couple with a cannula positioned over at least a portion of the adapter,
wherein the adapter is interchangeable between a plurality of adapters configured to couple with a variety of different cannulas.

2. The assembly of claim 1, wherein the adapter has one or more latching connectors at a proximal end of the adapter, the adapter coupling with the housing at least in part via the latching connectors.

3. The assembly of claim 2, wherein the housing comprises one or more openings configured to lockingly receive the one or more latching connectors therethrough.

4. The assembly of claim 1, wherein the housing has a recessed portion in the outer surface that is configured to receive at least a portion of a flange of the adapter, the flange disposed proximal of the cannula connector.

5. The assembly of claim 4, wherein the flange is configured to extend into the recessed portion so that an outer surface of the flange is flush with the outer surface of the housing adjacent the recessed portion.

6. The assembly of claim 1, further comprising a gasket interposed between a surface of the housing and a surface of the adapter, the gasket configured to seal the adapter relative to the tube of the endoscope.

7. The assembly of claim 1, wherein the cannula connector comprises a circumferential groove defined on an outer surface of the adapter, the groove configured to removably lock with a cannula when the cannula connector is inserted in a proximal end of the cannula.

8. An endoscope kit, comprising:
an endoscope comprising an endoscope tube;
a housing configured to extend over at least a portion of the endoscope so that the endoscope tube extends through and opening in the housing; and
a plurality of interchangeable adapters configured to be coupled to the housing, each adapter comprising:
a bore configured to receive the endoscope tube therethrough, and
a cannula connector on an outer surface of the adapter that is configured to couple with a cannula positioned over at least a portion of the adapter,
wherein the endoscope tube is configured to extend through the bore of the adapter, at least two of the plurality of interchangeable adapters being configured to lockingly couple with a different cannula.

9. The kit of claim 8, wherein each of the plurality of interchangeable adapters are configured to lockingly couple with a different cannula.

10. The kit of claim 9, wherein at least one of the plurality of interchangeable adapters is removably coupleable to the housing.

11. The kit of claim 8, wherein the housing has a recessed portion configured to receive at least a portion of the adapter therein.

12. The kit of claim 11, wherein the adapter has a flange adjacent the cannula connector configured to at least partially extend into the recessed portion when the adapter is coupled to the housing.

13. The kit of claim 8, wherein the housing has one or more openings configured to receive one or more latching connectors of the adapter therein to lockingly couple the adapter to the housing.

14. The kit of claim 8, further comprising a gasket interposed between a surface of the housing and a surface of the adapter, the gasket configured to seal the adapter relative to the tube of the endoscope.

15. The kit of claim 8, wherein the cannula connector comprises a circumferential groove defined on an outer surface of the adapter, the groove configured to removably lock with a proximal key connector of the cannula when the cannula connector is inserted into a proximal end of the cannula.

16. An interchangeable adapter for an endoscope assembly, comprising:
an adapter body configured to couple to a housing of the endoscope assembly, the adapter having a bore configured to receive an endoscope tube therethrough, the adapter having a cannula connector on an outer surface of the adapter that is configured to removably couple with a cannula when the cannula is positioned over at least a portion of the adapter.

17. The adapter of claim 16, further comprising means for coupling the adapter body with the housing.

18. The adapter of claim 16, further comprising one or more latching connectors at a proximal end of the adapter body, the adapter body configured to couple with the housing at least in part via the latching connectors.

19. The adapter of claim 16, wherein the adapter body further comprises a flange disposed proximal of the cannula connector.

20. The adapter of claim 19, wherein the flange is configured to extend into a recessed portion of the housing so that an outer surface of the flange is flush with the outer surface of the housing adjacent the recessed portion.
